(19) United States
(12) Patent Application Publication
(10) Pub. No.: US 2008/0031405 A1
(43) Pub. Date: Feb. 7, 2008

(54) IMAGE PROCESSING METHOD AND COMPUTER READABLE MEDIUM FOR IMAGE PROCESSING

(75) Inventor: Kazuhiko Matsumoto, Tokyo (JP)

Correspondence Address: PEARNE & GORDON LLP, 1801 EAST 9TH STREET, SUITE 1200, CLEVELAND, OH, 44114-3108, US

(73) Assignee: Ziosoft, Inc., Tokyo (JP)

(21) Appl. No.: 11/831,346

(22) Filed: Jul. 31, 2007

(30) Foreign Application Priority Data
Aug. 1, 2006 (JP) 2006-209831

(51) Int. Cl. A61B 6/03

(52) U.S. Cl. 378/8

(57) ABSTRACT

In MIP images in a related art, an image is rendered separately using volume data of each phase, whereby an almost still organ and blood vessel portions through which a contrast medium is passing are rendered separately for each phase. On the other hand, an MIP image according to an image processing method of the invention is rendered as one image by using the volume data of a plurality of phases, so that the whole of the blood vessel through which a contrast medium is passing can be displayed. Accordingly, the state of change in the blood stream can be displayed in one image, and the image can be put to work on precise diagnosis.

This application claims foreign priority based on Japanese Patent application No. 2006-209831, filed Aug. 1, 2006, the contents of which are incorporated herein by reference in its entirety.

### BACKGROUND OF THE INVENTION

1. Field of the Invention

This invention relates to an image processing method and a computer readable medium for image processing, for executing volume rendering using volume data.

2. Description of the Related Art

Hitherto, a three-dimensional image data provided as a volume data by a computed tomography (CT) apparatus, a magnetic resonance imaging (MRI) apparatus, and etc. Volume data has been projected in any desired direction to provide a projection image. Volume rendering is widely used as processing for providing such a projection image. As the volume rendering, for example, MIP (Maximum Intensity Projection) processing for extracting the maximum voxel value on a virtual ray relative to the projection direction to perform projection, MinIP (Minimum Intensity Projection) processing for extracting the minimum voxel value on a virtual ray to perform projection, a ray casting method of projecting a virtual ray in the projection direction and calculating reflected light from the object, and the like are known.

FIGS. 14A and 14B are schematic drawings of MIP image calculation in a related art. As an MIP image, a virtual ray 156 is projected to volume data 151 and the maximum value of voxel values on the virtual ray 156 is selected as display data. That is, if the voxel value of a voxel 152 is 1, the voxel value of a voxel 153 is 5, the voxel value of a voxel 154 is 3, and the voxel value of a voxel 155 is 1, the maxim value on the virtual ray 156, which is 5, is adopted as a display data of the pixel.

In recent years, the performance of a CT apparatus, etc., has been dramatically enhanced, so that it has been made possible to acquire volume data of a plurality of phases provided by scanning the same object according to the same technique at once, as time series data. To observe the blood stream in an organ with a CT apparatus, contrast medium is injected into a blood vessel as a marker and the process of inflow and outflow of the contrast medium is scanned in time series as a plurality of phases. The term “phase” is used to mean one set of data among the volume data provided by performing scanning on the same object according to a unitary method in a short time. For example, there exist volume data of a plurality of phases in time series and volume data of a plurality of phases for each contraction stage in contraction period of an organ such as a heart. Volume data of each phase may be data provided by synthesizing two or more scanning results along the cycle.

FIGS. 15A-15C show MIP images for volume data of a plurality of phases provided at one time by scanning the same object according to the same technique. The images are provided by scanning an organ 161 and blood vessel portions 162 to 165 at the same location at different timings and performing MIP processing of the volume data of the phases. That is, FIG. 15A shows an image created by performing MIP processing of the volume data of phase 1; FIG. 15B shows an image created by performing MIP processing of the volume data of phase 2; and FIG. 15C shows an image created by performing MIP processing of the volume data of phase 3.

Thus, in the MIP processing in the related art, if a plurality of phases exists, an image is rendered separately for each of the phases and an MIP image for each phase is displayed. According to the images, a part of the blood vessel through which blood containing a contrast medium flows in each phase is rendered in each image, so that how the contrast medium passes through the blood vessel can be displayed in time series.

FIG. 16 is a flowchart of the MIP method in the related art. In the MIP method in the related art, first, projection plane Image [p, q] is set (step S51), volume data Vol [x, y, z] is acquired (step S52), and double loop processing is started in p, q scanning over the projection plane to create an image (step S53).

Next, projection start point O (x, y, z) corresponding to p, q is set (step S54), a virtual ray is projected to volume data from O (x, y, z), and maximum value M of the voxel values on the virtual ray is acquired (step S55). The pixel value is calculated using the maximum value M and is adopted as the pixel value of Image [p, q] (step S56). Then, the process returns to step S53 and the processing is repeated.

Thus, in the MIP processing in the related art, if a plurality of phases exist, an image is rendered separately for each of the phases and thus the user needs to compare the images corresponding to the phases, so it is hard for the user to keep track of a tissue changing with time, such as the state of the whole blood vessel through which a contrast medium is passing.

### SUMMARY OF THE INVENTION

The present invention has been made in view of the above circumstances, and provides an image processing method and a computer readable medium for image processing capable of rendering a plurality of phases as one image.

In some implementations, an image processing method of the invention by volume rendering, the image processing method comprising:

acquiring volume data of a plurality of phases where the volume data is acquired by performing scanning on a same object in an unitary method for each of the phases;

projecting a virtual ray of the same trajectory to each of the phases;

acquiring a value of at least one point on the virtual ray among the plurality of phases, where the value is mutually exchangeable with that of another point on the virtual ray in determining a pixel value; and

determining the pixel value using the acquired value of said at least one point.

According to the configuration described above, since each pixel value is determined using the volume data of a plurality of phases acquired at one time by scanning the same object in an unitary method, a plurality of phases can be rendered as one image like an image into which images of the blood stream picked up at certain time intervals are synthesized, for example. Therefore, the state of change in the observation object can be grasped with one image.

In the image processing method of the invention, the virtual ray is projected independently to each other to the volume data of each of the plurality of phases,

the value of said at least one point on the virtual ray is acquired for each of the virtual ray, and

the pixel value is determined via the values acquired for each of the virtual ray.

According to the configuration described above, the position of the imaging object is fixed, and rendering is executed independently for the whole blood stream, etc., so that a plurality of phases can be displayed as one image. Further, since the voxel calculation order does not affect the result, rendering can be executed separately for each of a plurality of phases and the processing time can be easily shortened as parallel processing is performed.

In the image processing method of the invention, the virtual ray is projected to the volume data of the plurality of phases, the virtual ray being common for the volume data of the plurality of phases,

where, said at least one point is a single point, and the value of said one point has a maximum value on the common virtual ray, and

the pixel value is determined using the value of said one point having the maximum value on the common virtual ray.

In the image processing method of the invention, the virtual ray is projected to the volume data of the plurality of phases, the virtual ray being common for the volume data of the plurality of phases,

where, said at least one point is a single point, and the value of said one point has a minimum value on the common virtual ray, and

the pixel value is determined via the value of said one point having the minimum value on the common virtual ray.

According to the configuration described above, in the MIP method and the MinIP method, the voxel calculation order does not affect the result. Thus, the volumes can be arranged on a virtual space so as to match the projection direction of the virtual ray for calculation. Further, if the maximum value or the minimum value on the virtual ray is saturated, the later calculation can be early terminated, so that high-speed processing can be executed.

The image processing method of the invention further comprising:

synthesizing the volume data of the plurality of phases,

wherein the virtual ray is projected to the synthesized volume data.

According to the configuration described above, the synthesizing processing of a plurality of phases can be performed in combination with the MIP processing to render the images of the phases as one image, so that the object moving with time, such as blood containing a contrast medium, can be displayed as a whole. Since rendering after synthesis is calculation for one volume, rendering can be executed at high speed.

The image processing method of the invention further comprising:

performing registration of the plurality of phases based on a moving amount of the region data.

According to the configuration described above, the positions of the heart, etc., in a plurality of phases differ and the blood vessel portions through which blood containing a contrast medium flows differ, motion compensation is executed according to an registration algorithm, whereby rendering can be executed by compensating for the motion of the heart and the blood vessel.

The image processing method of the invention further comprising:

specifying the volume data of a predetermined phase from the volume data of the plurality of phases,

wherein the volume data of the specified phase is excluded from calculation in acquiring the value of said at least one point on the virtual ray and determining the pixel value using the acquired value.

The image processing method of the invention further comprising:

specifying predetermined region from the volume data of the plurality of phases,

wherein the specified region is excluded from calculation in acquiring the value of said at least one point on the virtual ray and determining the pixel value using the acquired value.

According to the configuration described above, if a noise component is superposed on a predetermined phase, the phase is excluded from calculation and if a noise component is superposed on predetermined region data, the region data is excluded from calculation, and then rendering is executed, whereby an organ and a blood vessel with the noise component removed can be rendered.

In the image processing method of the invention, the pixel value is determined by using a maximum value, a minimum value, an average value or an accumulation value of the values of said at least one point.

The image processing method of the invention is an image processing method wherein parallel processing is performed.

The image processing method of the invention is an image processing method wherein processing is performed by a GPU (Graphic Processing Unit).

The image processing method of the invention is an image processing method wherein a number of said at least one point is one, and the value of said one point on the virtual ray is acquired.

In some implementations, a computer readable medium storing a program including instructions for permitting a computer to execute image processing by volume rendering, the instructions comprising:

acquiring volume data of a plurality of phases where the volume data is acquired by performing scanning on a same object in an unitary method for each of the phases;

projecting a virtual ray of the same trajectory to each of the phases;

acquiring a value of at least one point on the virtual ray among the plurality of phases, where the value is mutually exchangeable with that of another point on the virtual ray in determining a pixel value; and

determining the pixel value using the acquired value of said at least one point.

According to the image processing method and the computer readable medium for image processing of the invention, a plurality of phases can be rendered as one image.

### BRIEF DESCRIPTION OF THE DRAWINGS

In the accompanying drawings:

FIG. 1 is a drawing to schematically show a computed tomography (CT) apparatus used with an image processing method according to one embodiment of the invention;

FIGS. 2A-2D are drawings to describe an outline of MIP processing conforming to the image processing method of the embodiment of the invention;

FIGS. 3A-3C are drawings to show the case where rendering is executed separately for each of a plurality of phases in the image processing method of the embodiment of the invention;

FIG. 4 is a flowchart of the image processing method of the embodiment of the invention (1);

FIG. 5 is a flowchart of the image processing method of the embodiment of the invention (2);

FIGS. 6A-6C are drawings to show the case where a common virtual ray is allowed to pass through a plurality of phases and rendering is executed in the image processing method of the embodiment of the invention;

FIGS. 7A-7E are drawings to show the case where a plurality of phases are synthesized before rendering is executed in the image processing method of the embodiment of the invention;

FIGS. 8A-8D are schematic representations for supporting explanation of an registration step in the image processing method of the embodiment of the invention (1);

FIGS. 9A-9D are schematic representations of an registration step in the image processing method of the embodiment of the invention (2);

FIG. 10 is a flowchart of an registration algorithm in the image processing method of the embodiment of the invention;

FIGS. 11A-11D are drawings for supporting explanation of the case where rendering is executed with an inappropriate phase removed in the image processing method of the embodiment of the invention (1);

FIGS. 12A-12D are drawings to show the case where rendering is executed with an inappropriate phase removed in the image processing method of the embodiment of the invention (2);

FIGS. 13A-13E are drawings to show the case where rendering is executed with a part of an inappropriate phase removed in the image processing method of the embodiment of the invention;

FIGS. 14A and 14B are schematic drawings of MIP image calculation in a related art;

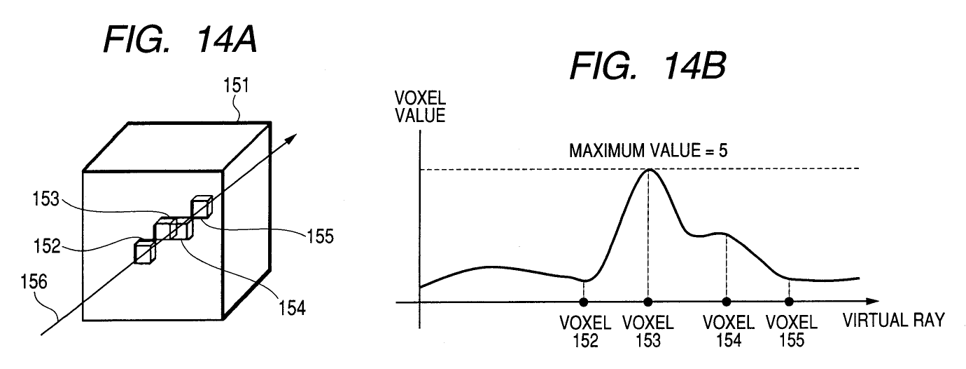

FIGS. 15A-15C are drawings to show MIP images in the related art for volume data of a plurality of phases; and

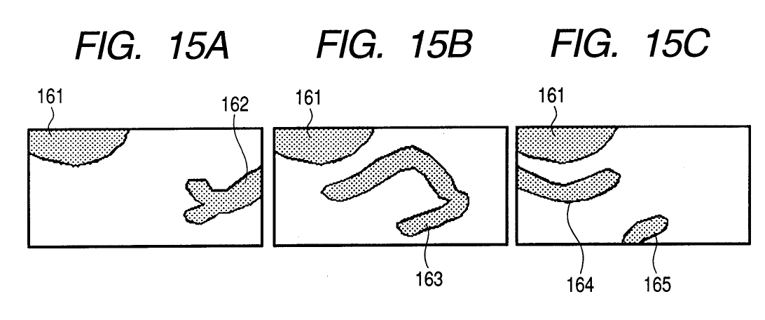

FIG. 16 is a flowchart of the MIP method in the related art.

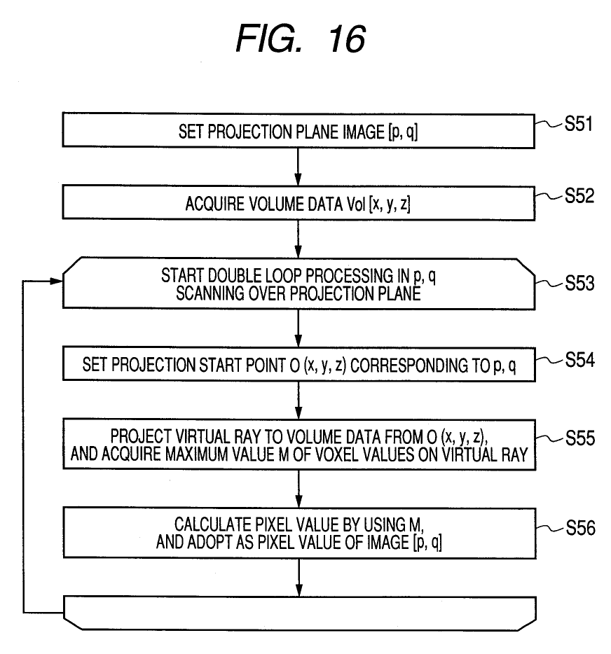

### DESCRIPTION OF THE PREFERRED EMBODIMENTS

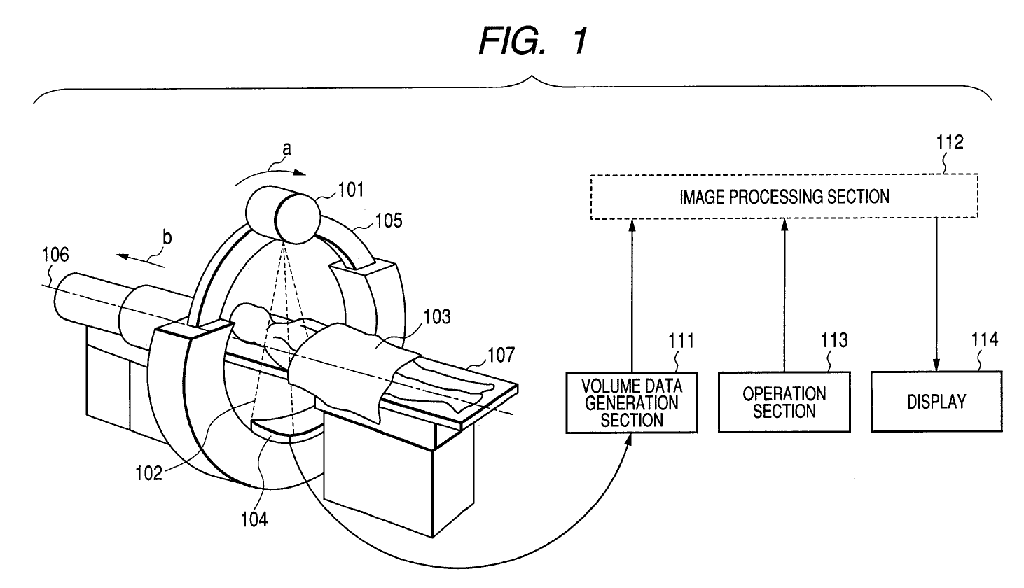

FIG. 1 schematically shows a computed tomography (CT) apparatus used with an image processing method according to one embodiment of the invention. The computed tomography apparatus is used for visualizing tissues, etc., of a subject. A pyramid-like X-ray beam 102 having edge beams which is represented by dotted lines in FIG. 1 is emitted from an X-ray source 101. The X-ray beam 102 is applied on an X-ray detector 104 after transmitting through the subject, for example, a patient 103. In this embodiment, the X-ray source 101 and the X-ray detector 104 are disposed in a ring-like gantry 105 so as to face each other. The ring-like gantry 105 is supported by a retainer not shown in FIG. 1 so as to be rotatable (see the arrow “a”) about a system axis 106 which passes through the center point of the gantry.

In this embodiment, the patient 103 is lying on a table 107 through which the X-rays are transmitted. The table 107 is supported by a retainer which is not shown in FIG. 1 so as to be movable (see the arrow “b”) along the system axis 106.

Thus a CT system is configured so that the X-ray source 101 and the X-ray detector 104 are rotatable about the system axis 106 and movable along the system axis 106 relatively to the patient 103. Accordingly, X-rays can be cast on the patient 103 at various projection angles and in various positions with respect to the system axis 106. An output signal from the X-ray detector 104 when the X-rays are cast on the patient 103 are supplied to a volume data generation section 111 and transformed into a volume data.

In sequence scanning, the patient 103 is scanned in accordance with each sectional layer of the patient 103. When the patient 103 is scanned, while the X-ray source 101 and the X-ray detector 104 rotate around the patient 103 about the system axis 106 as its center, the CT system including the X-ray source 101 and the X-ray detector 104 captures a large number of projections to scan each two-dimensional sectional layer of the patient 103. A tomogram displaying the scanned sectional layer is reconstructed from the measured values acquired at that time. While the sectional layers are scanned continuously, the patient 103 is moved along the system axis 106 every time the scanning of one sectional layer is completed. This process is repeated until all sectional layers of interest are captured.

On the other hand, during spiral scanning, the table 107 moves along the direction of the arrow “b” continuously while the CT system including the X-ray source 101 and the X-ray detector 104 rotates about the system axis 106. That is, the CT system including the X-ray source 101 and the X-ray detector 104 moves on a spiral track continuously and relatively to the patient 103 until the region of interest of the patient 103 is captured completely. In this embodiment, signals of a large number of successive sectional layers in a diagnosing area of the patient 103 are supplied to a volume data generation section 111 by the computed tomography apparatus shown in FIG. 1.

Volume data generated by the volume data generation section 111 is introduced into an image processing section 112. The image processing section 112 performs volume rendering using the volume data to generate a projection image. The projection image generated by the image processing section 112 is supplied to and is displayed on a display 114. Additionally, histograms may be overlaid with the projection image, and plurality of images may be displayed parallel with the projection image, such as, animation of phases and simultaneous display with a virtual endoscopic (VE) image.

An operation section 113 contains a GUI (Graphical User Interface) and sets an image processing method, etc., in response to operation signals from a keyboard, a mouse, etc., and generates a control signal of each setup value and supplies the control signal to the image processing section 112. Accordingly, the user can interactively change the image and observe the lesion in detail while viewing the image displayed on the display 114.

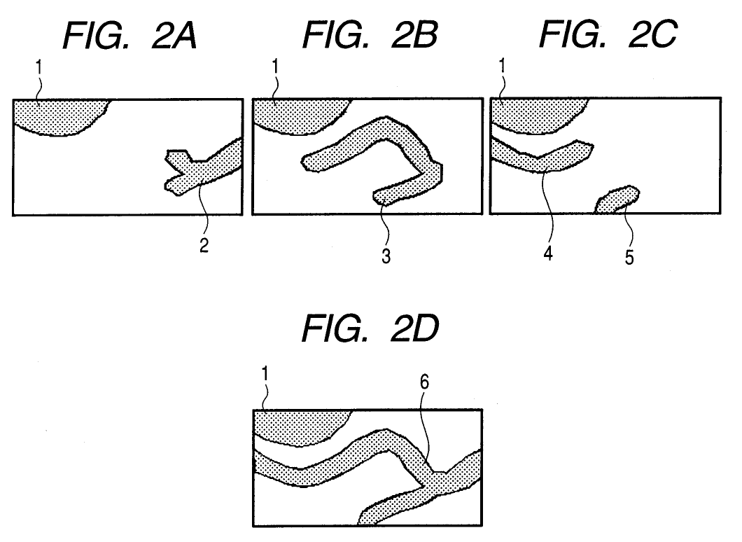

FIGS. 2A-2D are drawings to describe an outline of MIP processing conforming to the image processing method of the embodiment. In the embodiment plurality of phases are included in a volume data. FIGS. 2A-2C show MIP images in the related art, where images are rendered separately using each of phases 1, 2, and 3 contained in the volume data. A still organ 1 and blood vessel portions 2, 3, 4, and 5 through which a contrast medium passes are rendered separately for each of the phases.

On the other hand, as shown in FIG. 2D, the MIP image according to the image processing method of the embodiment is rendered as one image using volume data of a plurality of phases, so that the whole of a blood vessel 6 through which a contrast medium passes can be displayed. Accordingly, the image can be represented as an image into which images of the blood stream scanned at certain time intervals are synthesized, and the image can be put to work on precise diagnosis.

### EXAMPLE 1

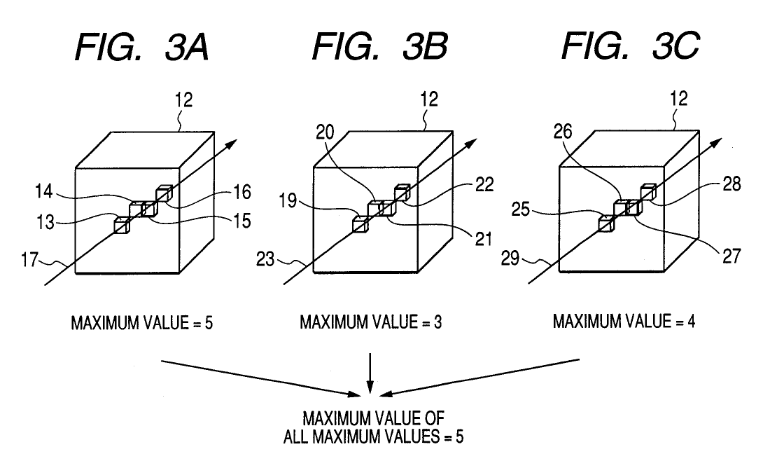

FIGS. 3A-3C show the case where rendering is executed independently for each of a plurality of phases included in the volume data in the image processing method of the embodiment. In the embodiment, to determine the final pixel value, a virtual ray is projected independently to each of a plurality of phases included in the volume data, the maximum value of voxels on the virtual ray in each of the phases is acquired, and the maximum value of all the maximum values of the phases is used as the pixel value.

For example, a virtual ray 17 of the same trajectory is projected to phase 1 of the volume data 12 shown in FIG. 3A from the same coordinates on the projection plane to acquire the maximum value 5 of a voxel 13, a virtual ray 23 of the same trajectory is projected to phase 2 included in the volume data 12 shown in FIG. 3B from the same coordinates on the projection plane to acquire the maximum value 3 of a voxel 20, and a virtual ray 29 of the same trajectory is projected to phase 3 included in the volume data 12 shown in FIG. 3C from the same coordinates on the projection plane to acquire the maximum value 4 of a voxel 28. The maximum value 5 of all of the maximum values of the phases 1, 2, and 3 is used as the pixel value on the projection plane for the volume data 12.

In the usual MIP method and MinIP method, one of voxel values on a single virtual ray is selected and is used as the pixel value. In the image processing method of the embodiment, however, one of voxel values on a plurality of virtual rays projected to a plurality of phases included in the volume data from the same point of image is selected and is used as the pixel value as described above. In the RaySum method or the average value method in related arts, the sum or the average value of voxel values on virtual ray is used as the pixel value. In the image processing method of the embodiment, however, the sum or the average value of voxel values on a plurality of virtual rays projected to a plurality of phases included in the volume data from the same point of image is used as the pixel value.

According to the embodiment, the location of the imaging object is fixed and rendering is executed separately for the whole blood stream, so that a plurality of phases can be displayed as one image. Additionally, rendering is executed independently for a plurality of phases, so that the processing of each phases can be paralleled and rendering time can be shortened.

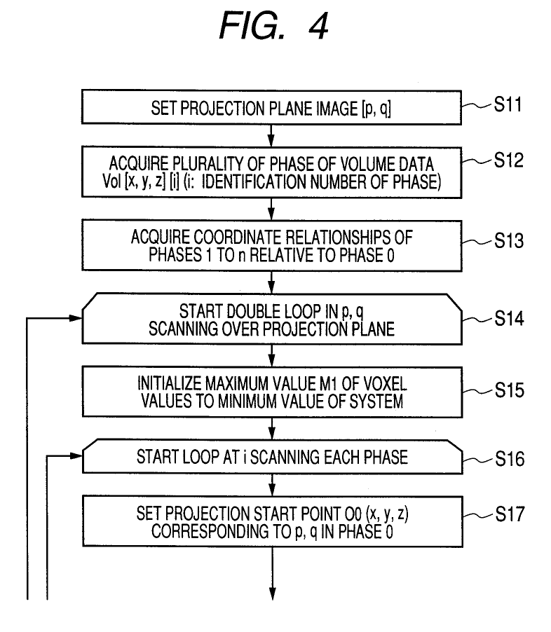

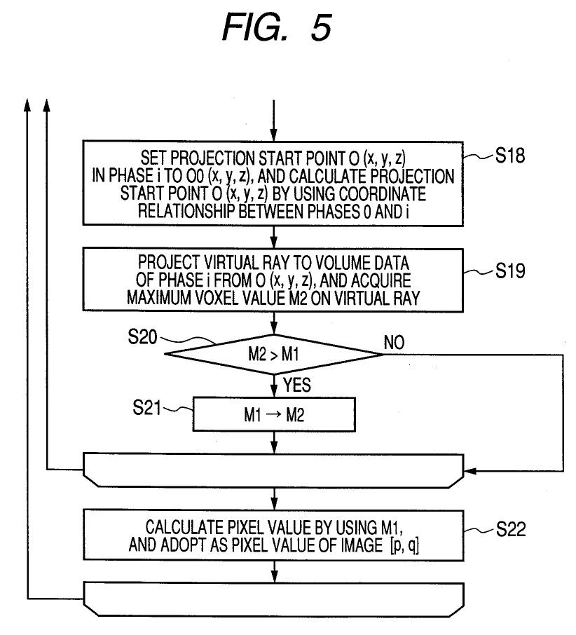

FIGS. 4 and 5 are flowcharts of the image processing method of the embodiment. In the embodiment, first, projection plane Image [p, q] is set (step S11), a plurality of phases included in the volume data Vol [x, y, z] [i] (i: Identification number of phase) (step S12), and the coordinate relationships of phases 1 to n relative to phase 0 are acquired (step S13).

Next, double loop is started in p, q scanning over the projection plane (step S14), and maximum value M1 of the voxel values is initialized to the minimum value of the system (step S15). Loop is started at i scanning each phase (step S16), and projection start point O0 (x, y, z) corresponding to p, q in phase 0 is set (step S17).

Next, the projection start point O (x, y, z) in phase is set to O0 (x, y, z), and is calculated using the coordinate relationship between phases 0 and i (step S18), and a virtual ray is projected to the phase i of the volume data from O (x, y, z) and maximum voxel value M2 on the virtual ray is acquired (step S19).

Next, a comparison is made between M2 and M1 (step S20) If M2>M1 (yes), processing of M1<-M2 is performed for replacing the maximum values of the volume data (step S21) and the process returns to step S16. Thus, at steps S14 to S21, the volume data of a plurality of phases with their coordinate relationship adjusted is used and the values of one or more points mutually exchangeable on the virtual ray are used to determine the pixel value. When the loop is completed, the pixel value is calculated using M1 and is adopted as the pixel value of Image [p, q] (step S22). Then, the process returns to step S14 and the processing is repeated.

### EXAMPLE 2

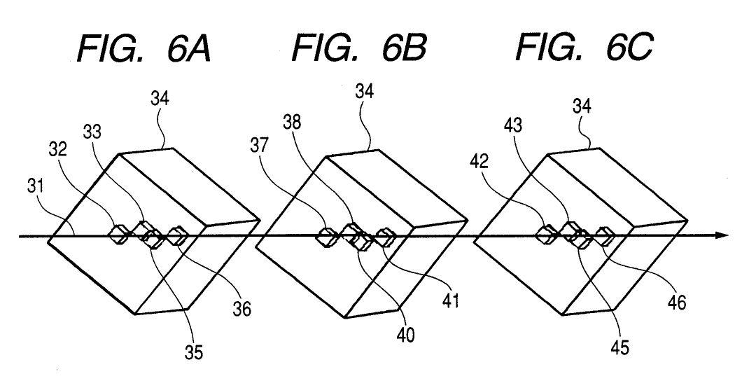

FIGS. 6A-6C show the case where a common virtual ray is allowed to pass through a plurality of phases of the volume data and rendering is executed in the image processing method of the embodiment. In the embodiment, a common virtual ray 31 is projected to volume data 34 of phase 1 shown in FIG. 6A, volume data 34 of phase 2 shown in FIG. 6B, and volume data 34 of phase 3 shown in FIG. 6C, whereby the virtual ray of the same trajectory is allowed to pass through the volume data, and the maximum values of the voxel values of all of the phases 1, 2, and 3 is used as the pixel value for the volume data 34.

In the MIP method and the MinIP method, unlike the ray casting method considering the light amount attenuation of the virtual ray, the same image is obtained even if the volume data is inverted in the depth direction. This also applies to the RaySum method and the average value method, because only operations are used in which mathematical commutation rule is ensured. The result is stable even if the values making up the accumulation value or the average value are swapped. Thus, a plurality of voxel values on the corresponding coordinates in a plurality of phases included in the volume data can be represented as the values of one or more points whose positional relationship on the virtual ray can be replaced with each other.

The commutation rules also hold, for example, in case in which the average value of the high-order 10 values on the virtual ray is displayed as a pixel value.

Thus, in the MIP method, the MinIP method, the RaySum method, and the average value method, the voxel calculation order does not affect the result. Therefore, a plurality of phases in a volume data can be arranged on a virtual space so as to match the projection direction of the virtual ray for calculation. Accordingly, for example, in the MIP method and the MinIP method, if the maximum value or the minimum value on the virtual ray is saturated, calculation can be early terminated, so that high-speed processing can be performed.

The image processing method of the embodiment is effective in the MIP method, the MinIP method, the RaySum method, and the average value method using plurality of phases included in the volume data. Light amount attenuation of a virtual ray passing through each voxel is calculated in the ray casting method, but not calculated in the MIP method. Thus, the MIP method has a feature that even if a virtual ray is projected from an opposite direction, the result image does not change. Accordingly, the voxel value on the virtual ray acquired from volume data or the value provided by interpolating the voxel value can be represented as the value having mutually exchangeable positional relationship and if the voxel values on the virtual ray are swapped, the result image does not change.

### EXAMPLE 3

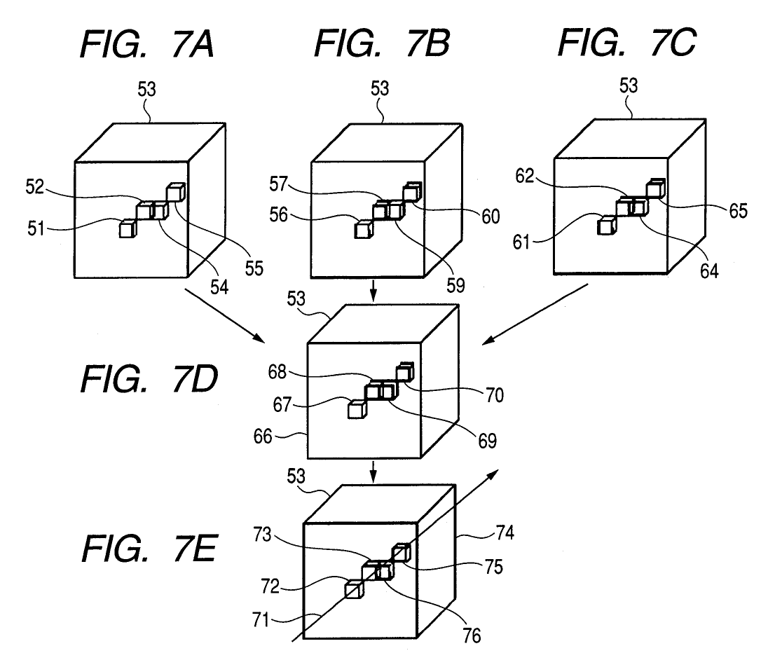

FIGS. 7A-7D show the case where a plurality of phases are synthesized before rendering is executed in the image processing method of the embodiment. That is, the coordinates of phase 1 in volume data 53 shown in FIG. 7A, phase 2 in volume data 53 shown in FIG. 7B, and phase 3 in volume data 53 shown in FIG. 7C are adjusted, and the phases included in volume data are superposed on each other and synthesized into a volume data of a single phase 53 shown in FIG. 7D. Then, a virtual ray 71 is projected to the volume data 53 as shown in FIG. 7E and rendering processing is performed. In so doing, the virtual ray of the same trajectory is allowed to pass through the volume data 53 of the phases.

In the embodiment, the synthesis processing of a plurality of phases can be performed in combination with the MIP processing to render the images of the phases as one image, so that the state of the observation object changing with time, such as blood containing a contrast medium, can be displayed as one image. Since rendering processing after synthesis is calculation for one volume, rendering can be executed at high speed and the memory amount can be saved. However, when registration between phases is changed, re-synthesizing is necessary.

### EXAMPLE 4

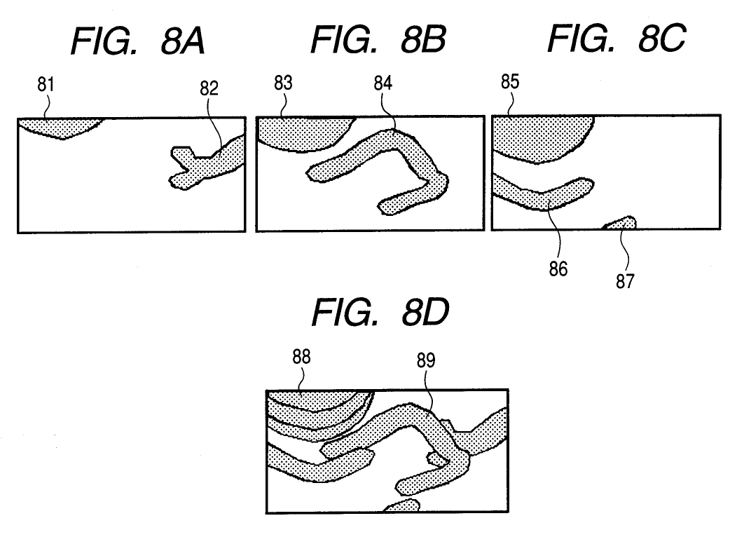

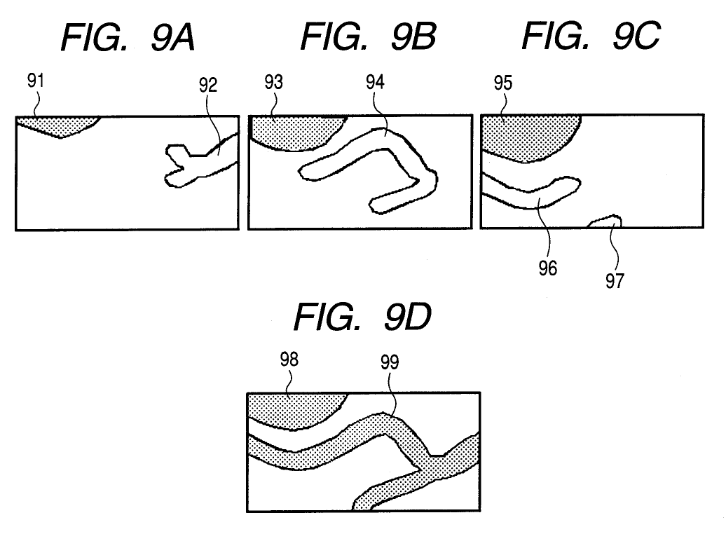

FIGS. 8A-8D and 9A-9D are schematic representations for explaining the case where motion compensation is added as a registration step in the image processing method of the embodiment. FIGS. 8A to 8C show hearts 81, 83, and 85 pulsating in phases 1 to 3 and blood vessel portions 82, 84, 86, and 87 through which blood containing a contrast medium flows in the phases 1 to 3. In this case, if rendering is executed using the phases 1 to 3 without executing motion compensation, a heart 88 and a blood vessel 89 are rendered as they shift, as shown in FIG. 8D. An existing Registration technique can be used to create a fusion image for registration of coordinates. In this example, registration technique obtained by extending the existing Registration technique is introduced. Since different parts on the blood vessel 89 of the observation object are imaged according to contrast medium in each of the phases, it is difficult to set a reference point for registration and thus the existing technique cannot be applied as it is.

FIGS. 9A-9D show the case where motion compensation is executed in the image processing method of the embodiment. As shown in FIGS. 9A to 9C, if hearts 91, 93, and 95 pulsate in phases 1 to 3 and blood vessel portions 92, 94, 96, and 97 through which blood containing a contrast medium flows differ, motion compensation is executed, whereby rendering can be executed by compensating for motion of a heart 98 and a blood vessel 99 as shown in FIG. 9D. This is important particularly for observing the heart. In addition, it is effective for observing an organ moving in response to respiration and pulsation of the heart. For example, the lungs contract at the respiration time and are affected by pulsation of the heart adjacent to the lungs.

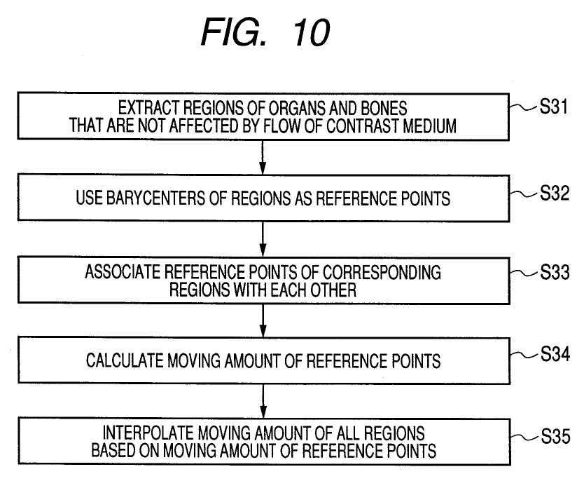

FIG. 10 is a flowchart of a motion compensation algorithm in the image processing method of the embodiment. In the algorithm, first the regions of organs and bones that are not affected by the flow of a contrast medium are extracted (step S31), and the barycenters of the regions are used as reference points (step S32). Next, the reference points of the corresponding regions are associated with each other (step S33), the moving amount of the reference points is calculated (step S34), and the moving amount of all regions is interpolated based on the moving amount of the reference points (step S35). The organs and bones that are not affected by the flow of a contrast medium can be extracted by identifying air in the lungs and calcium of the bones according to the pixel values and the shapes, for example.

According to the image processing method of the embodiment, in a case where the positions of the heart, etc., in a plurality of phases differ and the blood vessel portions through which blood containing a contrast medium flows differ, motion compensation is executed according to the motion compensation algorithm, whereby rendering can be executed by compensating for the motion of the heart and the blood vessel.

### EXAMPLE 5

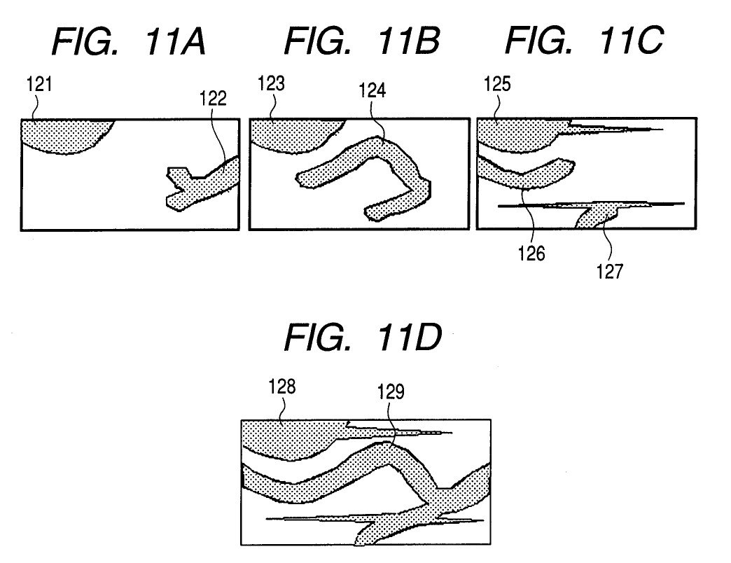

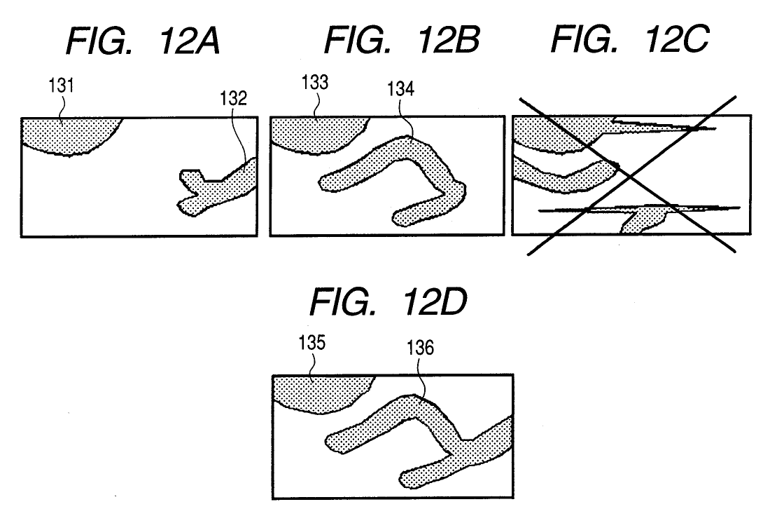

FIGS. 11A-11D and 12A-12D show drawings for the case where rendering is executed with an inappropriate phase removed in the image processing method of the embodiment. In scanning a plurality of phases, a noise component may be superposed on an organ 125 and a blood vessel portion 127 as shown in FIG. 11C because of a failure of electrocardiogram synchronization, etc., and an inappropriate phase may be generated. In this case, if rendering is executed using the maximum values of the phases 1 to 3 included in the volume data, an image with the noise component superposed on an organ 128 and a blood vessel 129 is displayed as shown in FIG. 11D.

FIGS. 12A-12D show the case where rendering is executed with an inappropriate phase removed in the image processing method of the embodiment. In the embodiment, if a noise component is superposed on phase 3 as shown in FIG. 12C, the phase 3 is excluded from calculation, rendering is executed using the maximum values of the voxel values of phases 1 and 2, and an organ 135 and a blood vessel 136 with the noise component removed can be rendered as shown in FIG. 12D.

In this case, the phase to be excluded from the calculation can be determined as follows: For example, (a) the user specifies an inappropriate phase. (b) An inappropriate phase is specified automatically. In this case, (1) the difference between the voxel values of the phase to be checked and another phase is acquired and (2) if the sum of the voxel value differences exceeds a given value, the phase is determined as an inappropriate phase. (c) An inappropriate phase is specified using external information of electrocardiogram information, etc., at the scanning time or the like.

### EXAMPLE 6

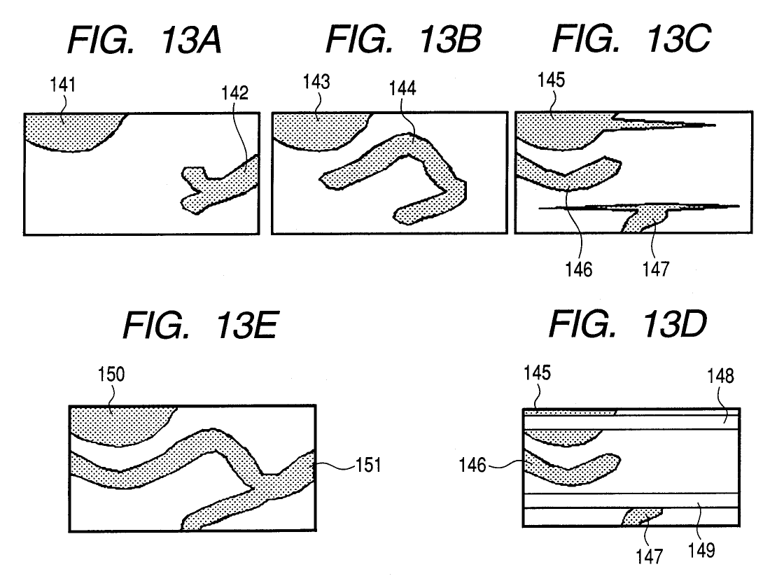

FIGS. 13A-13E shows the case where rendering is executed with a part of an inappropriate phase removed in the image processing method of the embodiment. That is, if noise is superposed on an organ 145 and a blood vessel portion 147 of phase 3 as shown in FIG. 13C, the voxels corresponding to image regions 148 and 149 where noise is displayed are excluded from the volume data of the phase 3 as shown in FIG. 13D. An organ 150 and a blood vessel 151 with the noise component removed can be rendered as shown in FIG. 13E based on the maximum values of the voxel values of phases 1 to 3.

Thus, if an inappropriate phase is generated, a part of the inappropriate phase rather than the whole of the inappropriate phase may be removed, because an inappropriate phase often occurs in only some slices of a volume in a medical image. A CT apparatus and an MRI apparatus perform scanning in slice units. For an apparatus for acquiring a plurality of slices at the same time, the slices acquired at the same time can be handled as one unit.

In this case, the region to be excluded from calculation can be determined as follows: For example, (a) the user specifies an inappropriate region. (b) An inappropriate phase is specified automatically. In this case, (1) the difference between the voxel values of the phase to be checked and another phase is acquired, (2) volume is divided into regions responsive to group of slices according to multi detector scanning, (3) the sum of the differences between the preceding and following phases is calculated for each region, and (4) if the sum exceeds a given value, the region is determined an inappropriate region. (c) An inappropriate region is specified particularly in slice units (because of scanning in slice units). (d) An inappropriate region is specified using external information of electrocardiogram information, etc., at the scanning time or the like.

The image processing method of the embodiment can be used in combination with a perfusion image. In the perfusion image, a flow rate of the blood stream in time series (a plurality of phases) is calculated by using a contrast medium, and the state of the contrast medium flowing in each image in time series is displayed. Using an MIP image according to the image processing method of the embodiment, all contrast media in time series would be able to be displayed. Therefore, if comparison observation with the perfusion image is conducted, it is effective. A plurality of phases may be grouped and an MIP image according to the image processing method of the embodiment may be calculated in each group. In so doing, reentry of a blood stream can be observed.

The perfusion image visualizes tissue perfusion dynamics. In many cases, the blood stream in an organ is visualized and congestion and loss of the blood stream can be observed. With a CT apparatus, a contrast medium is injected into a blood vessel as a marker and the process of inflow and outflow of the contrast medium is scanned as a moving image, the moving image is analyzed, and a perfusion image is created.

To generate an MIP image according to the image processing method of the embodiment, the used contrast medium amount can be reduced. To generate an MIP image according to the method in the related art, a large amount of contrast medium is used over all the scanning range. In contrast, to generate an MIP image according to the image processing method of the embodiment, the process in which a small amount of contrast medium extends into a body is scanned successively to create a plurality of volume data images, and observation with an MIP image can be conducted.

Thus, the number of scanning increases, but the radiation amount may be decreased. Although the image quality for each phase degrades as the radiation amount for each phase is decreased, an MIP image is created using a plurality of phases and consequently the S/N ratio is maintained and the image quality does not degrade as a whole.

Calculation processing of generating a projection image can be performed by a GPU (Graphic Processing Unit). The GPU is a processing unit designed for being specialized for image processing as compared with a general-purpose CPU, and usually is installed in a computer separately from the CPU.

In the image processing method of the embodiment, volume rendering calculation can be divided at predetermined angle units, image regions, volume regions, etc., and the divisions can be superposed later, so that the volume rendering calculation can be performed by parallel processing, network distributed processing, a dedicated processor, or using them in combination.

The embodiment of the invention can be also achieved by a computer readable medium in which a program code (an executable program, an intermediate code program, and a source program) according to the above described image processing method is stored so that a computer can read it, and by allowing the computer (or a CPU or an MCU) to read out the program (software) stored in the storage medium and to execute it.

The computer readable medium includes, for example, a tape-type medium, such as a magnetic tape or a cassette tape, a disc-type medium including a magnetic disc, such as a floppy® disc or a hard disc, and an optical disc, such as CD-ROM/MO/MD/DVD/CD-R, a card-type medium, such as an IC card (including a memory card) or an optical card, and a semiconductor memory, such as a mask ROM, an EPROM, an EEPROM, or a flash ROM.

Further, the computer may be constituted such that it can be connected to a communication network, and the program may be supplied thereto through the communication network. The communication network includes, for example, the Internet, the Intranet, an intranet, an extranet, a LAN, an ISDN, a VAN, a CATV communication network, a virtual private network, telephone lines, a mobile communication network, and a satellite communication network. A transmission medium for constituting the communication network includes, for example, wire lines, such as IEEE1394, USB, power lines, cable TV lines, telephone lines, and ADSL lines, infrared rays, such as IrDA or a remote controller, and wireless lines, such as Bluetooth®, 802.11 Wireless, HDR, a mobile communication network, satellite lines, and a terrestrial digital broadcasting network. In addition, the program may be incorporated into carrier waves and then transmitted in the form of computer data signals.

It will be apparent to those skilled in the art that various modifications and variations can be made to the described preferred embodiments of the present invention without departing from the spirit or scope of the invention. Thus, it is intended that the present invention cover all modifications and variations of this invention consistent with the scope of the appended claims and their equivalents.

What is claimed is:

1. An image processing method by volume rendering, the image processing method comprising:
acquiring volume data of a plurality of phases where the volume data is acquired by performing scanning on a same object in an unitary method for each of the phases;
projecting a virtual ray of the same trajectory to each of the phases;
acquiring a value of at least one point on the virtual ray among the plurality of phases, where the value is mutually exchangeable with that of another point on the virtual ray in determining a pixel value; and
determining the pixel value using the acquired value of said at least one point.

2. The image processing method as claimed in claim 1,
wherein the virtual ray is projected independently to each other to the volume data of each of the plurality of phases,
the value of said at least one point on the virtual ray is acquired for each of the virtual ray, and
the pixel value is determined via the values acquired for each of the virtual ray.

3. The image processing method as claimed in claim 1,
wherein the virtual ray is projected to the volume data of the plurality of phases, the virtual ray being common for the volume data of the plurality of phases,
where, said at least one point is a single point, and
the value of said one point has a maximum value on the common virtual ray, and
the pixel value is determined via the value of said one point having the maximum value on the common virtual ray.

4. The image processing method as claimed in claim 1,
wherein the virtual ray is projected to the volume data of the plurality of phases, the virtual ray being common for the volume data of the plurality of phases,
where, said at least one point is a single point, and the value of said one point has a minimum value on the common virtual ray, and
the pixel value is determined by using the value of said one point having the minimum value on the common virtual ray.

5. The image processing method as claimed in claim 1, further comprising:
synthesizing the volume data of the plurality of phases,
wherein the virtual ray is projected to the synthesized volume data.

6. The image processing method as claimed in claim 1, further comprising:
performing registration of the plurality of phases based on a moving amount of a region data.

7. The image processing method as claimed in claim 1, further comprising:
specifying the volume data of a predetermined phase from the volume data of the plurality of phases,
wherein the volume data of the specified phase is excluded from calculation in acquiring the value of said at least one point on the virtual ray and determining the pixel value via the acquired value.

8. The image processing method as claimed in claim 1, further comprising:
specifying a predetermined region from the volume data of the plurality of phases,
wherein the specified region is excluded from calculation in acquiring the value of said at least one point on the virtual ray and determining the pixel value via the acquired value.

9. The image processing method as claimed in claim 1, wherein the pixel value is determined by using a maximum value, a minimum value, an average value or an accumulation value of the values of said at least one point.

10. The image processing method as claimed in claim 1, wherein parallel processing is performed.

11. The image processing method as claimed in claim 1, wherein processing is performed by a GPU (Graphic Processing Unit).

12. The image processing method as claimed in claim 1, wherein a number of said at least one point is one, and the value of said one point on the virtual ray is acquired.

13. A computer readable medium storing a program including instructions for permitting a computer to execute image processing by volume rendering, the instructions comprising:
acquiring volume data of a plurality of phases where the volume data is acquired by performing scanning on a same object in an unitary method for each of the phases;
projecting a virtual ray of the same trajectory to each of the phases;
acquiring a value of at least one point on the virtual ray among the plurality of phases, where the value is mutually exchangeable with that of another point on the virtual ray in determining a pixel value; and
determining the pixel value using the acquired value of said at least one point.
